(19) United States
(12) Patent Application Publication
(10) Pub. No.: US 2009/0233868 A1
(43) Pub. Date: Sep. 17, 2009

(54) Small antiviral peptides against hepatitis C virus

(76) Inventors: Saumitra Das, Kolkata (IN); Siddhartha Roy, Kolkata (IN); Upasana Ray, New Delhi (IN); Asit Kumar Manna, East Midnapur (IN); Tanmoy Mondal, Birbhum (IN); Romi Gupta, Varanasi (IN); Renuka Pudi, Bangalore (IN); Sudhamani Sonny, Bangalore (IN)

Correspondence Address: LATHROP & GAGE LLP, 4845 PEARL EAST CIRCLE, SUITE 201, BOULDER, CO, 80301, US

(21) Appl. No.: 12/284,728

(22) Filed: Sep. 24, 2008

Related U.S. Application Data
(63) Continuation-in-part of application No. 11/913,311, now abandoned.

(30) Foreign Application Priority Data
May 2, 2005 (IN) 520/CHE/2005

(51) Int. Cl. A61K 38/16; C07K 14/00; C07K 7/00; C12N 15/11; C12N 15/00; A61K 38/10; A61K 38/08; A61K 31/7088

(52) U.S. Cl. 514/13; 530/325; 530/326; 530/327; 530/328; 530/329; 536/23.72; 435/320.1; 514/14; 514/15; 514/16; 514/44 R

(57) ABSTRACT

Disclosed herein is a small 7 amino-acid peptide, corresponding to the C terminus of RRM2 of the human La protein that binds to the IRES element of hepatitis C virus RNA and its derivatives. This disclosure demonstrates that this 7-mer interacts with the HCV IRES element both in vitro and in vivo and can compete against cellular La protein in binding to the HCV RNA. It is also shown here that this 7-mer peptide is able to inhibit HCV-IRES mediated translation in vivo which, in turn, leads to decreased viral replication.

### RELATED APPLICATIONS

This application is a continuation-in-part of U.S. patent application Ser. No. 11/913,311 filed Oct. 31, 2007, which claims priority to International Patent Application PCT/IN06/000141 filed Apr. 24, 2006, which claims priority to Indian Patent Application Serial No. 520/CHE/2005, filed May 2, 2005. All of the aforementioned applications are herein expressly incorporated by reference into this application.

### SEQUENCE LISTING

This application is accompanied by a sequence listing both on paper and in a computer readable form that accurately reproduces the sequences described herein.

### BACKGROUND

This disclosure pertains to the human La protein which is known to facilitate IRES-mediated translation of hepatitis C virus RNA by binding to the IRES element. More particularly, this disclosure relates to small peptide fragments located at the C terminus of the human La protein, and the use of these peptides and their variants, polynucleotides encoding the peptides, antibodies against the peptides or fragments thereof, as well as other agents capable of modulating the function of the peptides.

Infection of hepatitis C virus (HCV) has been shown to be the primary cause for non-A and non-B viral hepatitis, which may lead to development of chronic hepatitis, cirrhosis, or hepato-cellular carcinoma. Houghton et al., 1991. It is estimated that about 3% of the world population is infected with HCV and about 85% of infected individuals develop chronic infection.

HCV belongs to the Flaviviridae family, and is an enveloped virus having a positive-sense, single-stranded RNA as its genome. See Choo et al., 1989. The 9.6 kb long genome encodes a single polyprotein of about 3,000 amino acids. The polyprotein is processed by host cell and viral proteases into three major structural proteins and several non-structural proteins necessary for viral replication. Kato et al., 1990.

The translation of the positive stranded genomic RNA to produce the viral proteins required for replication is an early obligatory step of the infection process. The translation initiation of the uncapped viral RNA is mediated by the interaction of ribosome and cellular proteins with an internal ribosome entry site (IRES) located within the 5′ untranslated region (5′UTR). The translation of the viral RNAs is believed to be controlled by the binding of certain trans-acting cellular proteins with certain highly structured cis-acting RNA elements. For review, see Tsukiyama-Kohara et al., 1992. Human La autoantigen (originally detected in patients with Lupus Erythematosus) was one of the first IRES trans-acting factors (ITAFs) identified that has been shown to interact with Poliovirus IRES element.

Translation initiation of HCV occurs in a cap-independent manner wherein the ribosomes are recruited onto an internal ribosome entry site (IRES) located mostly within the 5′ untranslated region (UTR) and extending a few nucleotides into the coding region. Id. HCV IRES has been shown to form three complex stem-loops and a pseudoknot, which encompasses the initiator AUG codon. Although the HCV IRES binds to the 40S ribosomal subunit specifically and stably even in the absence of any initiation factors, efficient translation requires canonical initiation factors such as eIF2 and eIF3, and other non-canonical trans-acting cellular proteins including polypyrimidine-tract binding protein (PTB), La autoantigen, poly (rC) binding protein (PCBP), heterogeneous nuclear ribonucleoprotein L (p68). Recently, binding of a 25 kDa cellular protein (p25) to HCV IRES has been shown to be important for efficient translation initiation of HCV. p25 was originally suggested to be ribosomal protein S9 but was later identified as rpS5.

Human La protein is known to interact with HCV IRES and stimulate translation initiation both in vitro and in vivo. See, e.g., Ali et al., 2000; Das, et al., 1998; Izumi et al., 2004; Pudi et al., 2003. La protein has been shown to interact with both the 5′- and 3′-UTR of hepatitis C virus RNA. Sequestration of La in rabbit reticulocyte lysate (RRL) results in inhibition of HCV IRES mediated translation, which can be rescued by exogenous addition of purified La protein. Due to the critical role played by the La protein in HCV IRES translation, disruption of its interaction with HCV IRES becomes a candidate target for inhibiting HCV IRES activity. A 60-nucleotide RNA (I-RNA) from the yeast Saccharomyces cerevisiae which preferentially blocked HCV and Poliovirus IRES mediated translation appeared to inhibit the translation by virtue of its ability to bind La protein. Das et al., 1998. A synthetic peptide corresponding to N-terminal “La motif” of human La autoantigen has been shown to inhibit HCV IRES mediated translation, possibly by binding to other essential cellular proteins. Izumi et al., 2004.

Despite significant progress in battling the HCV endemic, current therapeutic options in treating HCV involve α-interferon alone or in combination with ribavirin. These treatments fail to achieve sustained virological response in majority of patients. See Choo et al., 1989. Moreover, because the low stability of these therapeutic peptides inside the cells, frequent injections are required which increases the cost of treatment.

### SUMMARY

The present disclosure provides improved therapeutics and treatment methods for HCV infection. More particularly, human La protein has been shown to interact with the HCV IRES element in vivo and that this interaction enhances the efficiency of viral RNA translation. It is hereby disclosed that certain peptides derived from the La protein may inhibit this function of the La protein.

The La protein has three putative RNA Recognition Motifs (RRM 1-3), of which RRM2 has been shown to bind with high affinity around the GCAC sequence near the initiator AUG and the binding induces a conformational change in the HCV IRES, which is critical for the internal initiation. See Pudi et al., 2004. This disclosure provides a novel approach to inhibit HCV IRES mediated translation using a 24-amino acid peptide derived from the C terminus region of RRM2 of La protein. This small peptide, termed LaR2C, has a sequence of KYKETDLLILFKDDYFAKKNEERK (SEQ ID NO. 1), and is capable of binding to IRES element of HCV RNA and competing against the binding of cellular La protein to the same region of the HCV RNA. The LaR2C peptide prevents the ribosome assembly on HCV IRES and inhibits the internal initiation of translation of HCV both in vitro and in vivo.

In another embodiment, this disclosure provides a novel antiviral agent comprising the peptide LaR2C which binds to the IRES element of the HCV RNA and effectively blocks the ribosome assembly on the HCV RNA. The LaR2C peptide may be used alone or in combination with other agents for treatment of a viral infection and particularly Hepatitis C viral infection.

In yet another embodiment, the present disclosure provides peptides that are even smaller than the LaR2C peptide, which binds to the HCV IRES in competition against the La protein and thus inhibits La protein mediated translation. More particularly, a 7 amino-acid peptide (“7-mer,” “N7,” or “LaR2C N7”) having the sequence of KYKETDL (SEQ ID NO. 2) derived from the C-terminus of RRM2 retains the inhibitory activity possessed by the larger LaR2C peptide.

NMR and CD spectroscopy studies reveal that this 7-mer retains preference for a turn structure in solution. RNA bound structure of this peptide also forms a type VIII β-turn. NMR spectroscopy of the HCV-IRES-peptide complex shows significant shifts at two residues, P2 (tyr) and P4 (glu) of the LaR2C peptide. The nomenclature of P2 and P4 are based upon the sequence of the LaR2C peptide, P2 being the second residue from the N-terminus of the LaR2C peptide, and P4 being 4^{th} from the N-terminus. Mutations at the corresponding residues in full-length La protein result in significant decrease in HCV RNA binding. It is also shown that mutation at P4 residue in LaR2C results in drastic decrease in the RNA binding ability of the peptide and consequent reduction in translation inhibitory activity. These results suggest that the P2 (tyr) and P4 (glu) residues of the LaR2C peptide are important for HCV RNA binding.

The N7 peptide is also capable of impairing the HCV-IRES function in vivo. When the N7 is tagged with a hexa-arginine tag and added to a Huh7 cell culture, the tagged peptide is capable of entering the Huh7 cells. Data are provided showing that this tagged peptide inhibits HCV RNA replication in the Huh7 cells.

In another aspect, a composition comprising an HCV inhibiting agent may be used to treat or prevent viral infection of a host, said HCV inhibiting agent being capable of competing against the La protein for binding to HCV IRES RNA and thereby inhibiting the translation of HCV RNA mediated by the La protein, wherein the IC50 for said inhibition is not more than 100 μM, or more preferably, from 10 μM to 50 μM. The HCV inhibiting agent may be a peptide, a polynucleotide, or an organic molecule. More preferably, the HCV inhibiting agent is a peptide having less than 24 amino acids. The peptide may have at least 70% identity with SEQ ID NO. 2. More preferably, the peptide has a sequence identical to SEQ ID NO. 2.

In one embodiment, the HCV inhibiting agent may be a polynucleotide that contains a nucleic acid sequence encoding a peptide having at least 70% identical to the sequence of SEQ ID NO. 2. More preferably, the polynucleotide contains a nucleic acid sequence encoding a peptide that has a sequence identical to SEQ ID NO. 2. In another aspect, the polynucleotide may be placed in an expression vector. The expression vector preferably contains a promoter regulating the expression of the peptide such that when the expression vector is introduced into a host, the peptide may be expressed which is capable of competing against the La protein for binding to HCV IRES RNA and thereby inhibiting the translation of HCV RNA mediated by the La protein.

The present disclosure also identify a turn structure possessed by LaR2C N7 peptide. A molecule having a turn structure similar to the turn structure exhibited in solution by the LaR2C N7 peptide may be used to effectively block the ribosome assembly on the HCV RNA mediated by the La protein. This molecule may be selected from the group consisting of a peptide, a polynucleotide, and an organic molecule, and can be mixed with other antiviral agents or inactive ingredients, such as a carrier, to be used in HCV treatment therapy.

Thus, according to the present disclosure, various compositions may be administered to a host, such as a human, or an animal, to inhibit or to prevent viral infection of the host. The composition may comprise an HCV inhibiting agent that is capable of competing against the La protein for binding to HCV IRES RNA and thereby inhibiting the translation of HCV RNA mediated by the La protein, wherein the IC50 for said inhibition is not more than 100 μM. In one aspect, the HCV inhibiting agent may be a peptide, a polynucleotide, or an organic molecule. When the HCV inhibiting agent is a peptide, it may be administered to the host as a peptide, or it may be administered to the host as a polynucleotide which is then transcribed and/or translated in the host into a peptide.

In summary, this disclosure provides a novel antiviral agents comprising the various peptide fragments derived from the human La protein. These peptides compete against the full-length La protein in binding to the IRES element of hepatitis C virus RNA and effectively block the ribosome assembly on the HCV RNA. These peptides and their derivatives may be used alone or in combination with other agents for treatment of any viral infection and particularly HCV infection.

### BRIEF DESCRIPTION OF THE DRAWINGS

FIG. 1 shows the results of an NMR analysis of HCV IRES RNA bound peptide.

FIG. 2 shows the effect of point mutation in La protein on HCV IRES binding.

FIG. 3 shows the effect of P4 point mutation in LaR2C peptide activity.

FIG. 4 shows the structural characterization of the LaR2C-N7 peptide.

FIG. 5 shows the effect of LaR2C-N7 on HCV IRES-mediated translation in vitro.

FIG. 6 shows the effect of arginine-tagged LaR2C-N7 on HCV IRES function in Huh7 cells.

FIG. 7 shows the effects of Tat-N7 on HCV IRES mediated translation in vitro and on HCV replication ex vivo.

### DETAILED DESCRIPTION

The present disclosure provides a method for using a therapeutic protein, or peptide for treating and/or preventing HCV infection in a subject, such as a human or an animal. The peptides or proteins of the present disclosure preferably comprise a partial sequence derived from the La protein. For purpose of this disclosure, these proteins or peptides may be collectively called La-derived peptides. Examples of such therapeutic protein or peptides may include the LaR2C 24-mer or the N7 7-mer.

In another embodiment, the therapeutic composition may contain a core peptide derived from the La protein and one or more accessory peptide that confers certain property to the core peptide. In one aspect, the accessory peptide may facilitate purification of the core peptide. In another aspect, the accessory peptide may confer upon the core peptide the capability of entering a mammalian cell. In yet another aspect, the accessory peptide may enhance the protein stability of the core peptide, or help target the core protein to certain organ or tissue of the subject. The core peptide(s) and the accessory peptide(s) may exist in the same protein or they may exist in different proteins.

The peptides of the present disclosure may be prepared by chemical synthesis known to those of skill in the art. The peptides may also be produced using an expression vector having a nucleotide sequence encoding the peptide(s) of choice. The nucleotide sequence may be operably linked to an appropriate promoter, enhancer, terminator, or other sequences capable of regulating the expression of the encoded peptide. The nucleotide sequence may also be operably linked to other functional sequences. In one aspect, such a functional sequence may be a sequence encoding a purification tag, to facilitate expression and purification of the peptides. In another aspect, such a functional sequence may encode an accessory peptide that confers upon the core peptide various properties that are beneficial for the therapeutic functionality of the core peptide, for example, by increasing the stability of the core peptide, or by facilitating the delivery of the core peptide to its therapeutic target tissue or organ in the body.

The core peptide and the accessory peptide may be linked, for example, by one or more peptide bonds. The accessory peptide may be immediately C-terminal or N-terminal to the core peptide. More than one accessory peptide can be used. The fusion protein containing the accessory peptide and the core peptide can contain additional amino acids to the C-terminal, N-terminal, or both.

The administration of the La-derived peptide is preferably accomplished in the form of a pharmaceutical composition comprising a La-derived peptide and a pharmaceutically acceptable diluent, adjuvant, carrier, or other inactive ingredients. The inactive ingredients may help stabilize the pharmaceutically active peptide. The La-derived peptides may be administered without or in conjunction with known surfactants or other therapeutic agents. The peptide may be formulated in saline or a physiological buffer.

Therapeutic compositions comprising La-derived peptides may be administered via different routes, and systemic administration is preferred. Systemic routes of administration may include oral, intravenous, intramuscular or subcutaneous injection (including into a depot, or contained in a capsule for long-term release), intraocular and retrobulbar, intrathecal, intraperitoneal (e.g. by intraperitoneal lavage), intrapulmonary (using powdered drug, or an aerosolized or nebulized drug solution), or transdermal.

In one embodiment, when given parenterally, La-derived peptides may be injected in doses ranging from 1 μg/kg to 100 mg/kg per day by weight of the subject, preferably at doses ranging from 0.1 mg/kg to 20 mg/kg per day. The treatment may continue by continuous infusion or intermittent injection or infusion, at the same, reduced or increased dose per day for, e.g., 1 to 3 days, and additionally as determined by the treating physician.

The peptide of the present disclosure may also be delivered and/or put into use at a target organ or tissue of the subject by using methods developed and generally available in the field of gene therapy. See e.g., Nazari and Joshi, Curr. Gene Ther. 2008 August; 8(4):264-72. For example, gene therapy may be employed to cause the expression of the core peptides in the liver which is the most common site of infection by HCV. In another aspect, RNA interference may be employed to render non-expression of certain La derived proteins, in order to inhibit HCV RNA translation and HCV replication. See e.g., Lee and Chiang, Curr. Gene Ther. 2008 August; 8(4):236-46.

Those skilled in the art can readily optimize effective dosages and administration regimens for therapeutic compositions comprising La-derived peptides, as determined by good medical practice and the clinical condition of the individual subject. Use of the HCV inhibiting agent disclosed herein to prepare a medicament for treating and/or preventing infection by HCV or other viruses is also contemplated.

The terms “protein,” “polypeptide,” and “peptide” may be used interchangeably in this disclosure, all of which refer to polymers of amino acids. In addition to the peptides explicitly disclosed herein, certain “conservative” substitutions may be made on these peptides without substantially altering the functionality of the peptides.

“Conservative” substitutions of one amino acid for another are substitutions of amino acids having similar structural and/or chemical properties, and are generally based on similarities in polarity, charge, hydrophobicity, hydrophilicity and/or the amphipathic nature of the residues involved. The substituting amino acids may include naturally occurring amino acids as well as those amino acids that are not normally present in proteins that exist in the nature.

By way of example, hydrophilic basic amino acids may include but are not limited to lysine, arginine, histidine, ornithine, diaminobutyric acid, citrulline, or para-amino phenylalanine. Polar acidic amino acids may include but are not limited to aspartic acid and glutamic acid. Hydrophilic neutral amino acids may include but are not limited to asparagine, glutamine, serine, threonine, tyrosine, hydroxyproline, or 7-hydroxy-tetrahydroisoquinoline carboxylic acid. Hydrophobic amino acids may include but are not limited to alanine, naphthylalanine, biphenylalanine, valine, leucine, isoleucine, proline, hydroxyproline, phenylalanine, tryptophan, methionine, glycine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentane carboxylic acid, 1-amino-1-cyclohexane carboxylic acid, amino-benzoic acid, amino-naphthyl carboxylic acid, 7-amino butyric acid, beta-alanine, difluorophenylalanine, fluorophenylalanine, nipecotic acid, aminobutyric acid, thienyl-alanine, t-butyl-glycine. As a general rule, as the similarity between the amino acids being substituted decreases, the likelihood that the substitution will affect activity increases.

“Derivative” may refer to a molecule that is generated through chemical or physical modification of another molecule. In the case of a protein, chemical modifications may include but are not limited to deletion, addition or substitution of amino acids, creation of fusion proteins or tagged proteins, glycosylation, phosphorylation, etc.

The phrase “HCV inhibiting agent” refers to an agent that is capable of binding to HCV IRES RNA and thereby competing against cellular protein such as the La protein, and inhibiting the translation of HCV RNA mediated by the La protein. For purpose of this disclosure, an HCV inhibiting agent is not the full-length La protein itself, nor does an HCV inhibiting agent retain all functionality of the full-length La protein. Rather, an HCV inhibiting agent retains only partial functions of the full-length La protein. Thus, for example, in the case of LaR2C, it retains the RNA binding activity of the full-length La protein but does not promote ribosomal assembly. Consequently, an HCV inhibiting agent such as LaR2C acts as a dominant negative by competing against the full-length La protein in binding to the limited number of binding sites on the IRES RNA. Preferably, the HCV inhibiting agent is derived from human full-length La protein or its homolog in other organisms.

The term “IC50” is generally used in the pharmaceutical field to indicate how much of a particular drug or other substance (inhibitor) is needed to inhibit a given biological process (or component of a process, i.e. an enzyme, cell, cell receptor or microorganism) by half. In other words, IC50 (also known as 50% IC) is the half maximal (50%) inhibitory concentration (IC) of a substance. In this disclosure, IC50 is used specifically to measure how much of a particular HCV inhibiting agent is needed to reduce the La mediated HCV RNA translation in half. In a preferred embodiment, the HCV inhibiting agent is a peptide.

The term “treatment” as used herein encompasses both prophylactic and therapeutic treatment. Treatment of mammals, including humans, is preferred.

After entry of a Hepatitis C virus (HCV) into a host cell, the translation of the positive-strand genomic RNA to produce viral proteins is an early obligatory step for successful infection by the HCV. The translation initiation of the uncapped HCV RNA takes place through the highly structured IRES element located in the 5′-UTR of the viral RNA. Thus, the process of IRES-mediated translation initiation is an attractive target for antiviral drug design.

In one aspect, the HCV inhibiting agents may include various peptide fragments derived from the La protein which may compete against the La protein in binding to the HCV RNA. These peptides may effectively block La protein mediated HCV translation and/or viral replication and may therefore be used to treat and/or to prevent Hep C infection. Examples of these peptides may include, but are not limited to, LaR2C and LaR2C N7.

The selective inhibition of HCV IRES-mediated mechanism by the LaR2C and LaR2C N7 peptides may be advantageous over other antiviral agents. In one aspect, as the interactions between host cellular proteins and a highly conserved region of the viral RNA is targeted, the chance of generation of viral escape mutants is very low. Approaches such as siRNA treatment (RNA silencing) has demonstrated rapid emergence of escape mutants in poliovirus. Although the rate of HCV replication is not as high as that of poliovirus, any sequence-specific antiviral molecule would exert a selection pressure for the generation of escape variants, unlike a strategy targeting host protein-viral RNA interactions.

In another aspect, because the peptide molecules are derived from an endogenous host protein, it is unlikely that any significant immune responses would ensue even if the peptides are administered prophylactically to patients.

In another aspect, as the binding of the cellular proteins is known to be dependent on the secondary structure, more stable derivatives and small molecule structural analogs of the peptide could be utilized. The use of smaller peptides or their derivatives may result in better stability thereby significantly increasing the effectiveness of treatment against Hepatitis C viral infection.

In yet another aspect, the 24 amino-acid LaR2C or the 7 amino-acid LaR2C N7 may be easier to administer because smaller peptides generally have higher permeability at the cell membrane. Moreover, the smaller peptides may be less expensive to prepare than the longer counterparts.

In one embodiment of the present disclosure, the turn structure identified here may be useful in designing therapeutic agents for treating Hepatitis C viral infection. Various molecules that possess such a turn structure may be capable of binding the HCV-IRES and may be used as an antiviral agent. Such agents may include, for example, peptides, organic molecules, polynucleotides or the mixture thereof.

Throughout this disclosure, certain terms may be used in either capital or small letters, in singular or plural forms. These different forms may be used interchangeably unless otherwise specified in this disclosure.

### EXAMPLES

The following examples are intended to provide illustrations of the application of the present disclosure. The following examples are not intended to completely define or otherwise limit the scope of the invention.

### Example 1

### NMR Spectroscopy of the HCV IRES RNA Bound Peptide Complex

NMR spectroscopy was used to identify amino acid residues important for recognition of RNA. Because the RNA was relatively long (18-383 nt of HCV IRES), obtaining the full structure of the RNA-peptide complex (1:1) would be extremely difficult. As an alternative approach, NMR spectrum of the 24-mer peptide was studied in the absence or presence of sub-stoichiometric amount of RNA. It was assumed that under fast exchange condition, the chemical shifts of peptide protons in the absence of RNA will be average of free and bound species (intensity of RNA protons will be insignificant due to sub-stoichiometric presence and broader line-width). Thus, comparison of peptide chemical shifts in the presence of RNA with that of the free peptide was expected to shed light on the residues that may be involved in recognition.

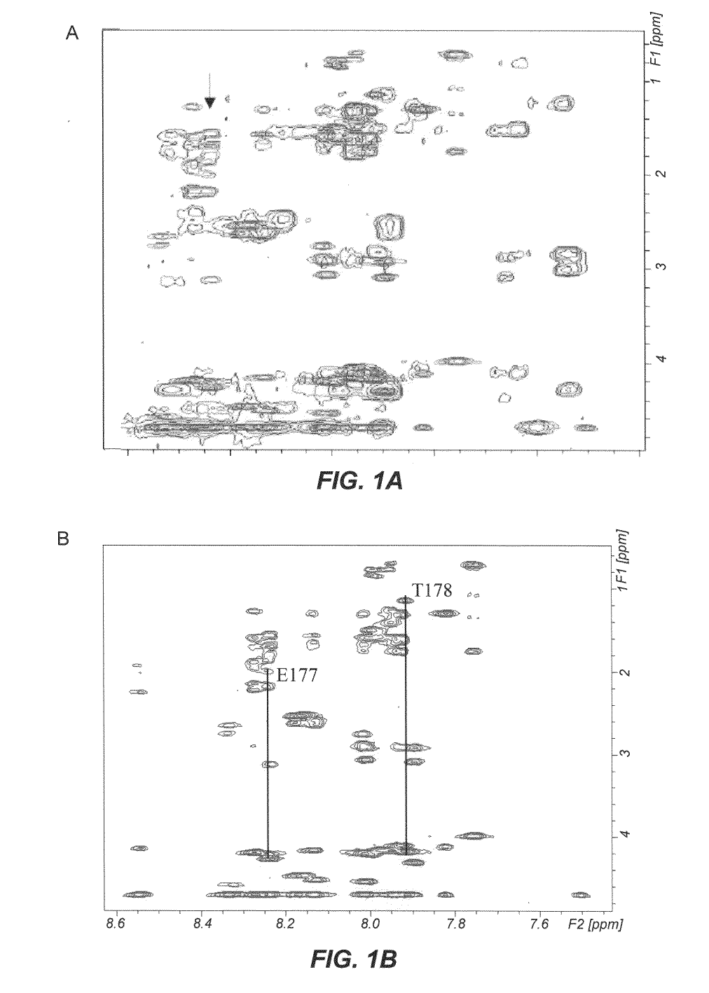

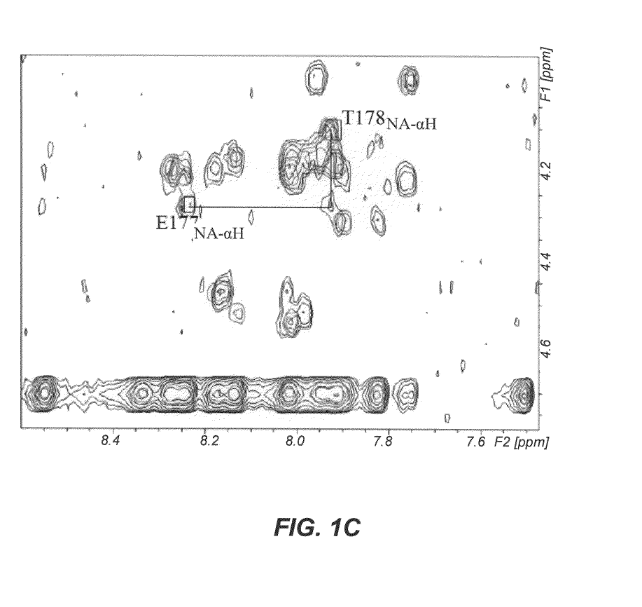

“Total Correlation Spectroscopy” (TOCSY) provides connectivity between all adjacent protons (three-bond connectivity) within an amino acid unit and hence a fingerprint for the identity of the amino acid. FIG. 1A shows the TOCSY spectrum of the region that connects NH (approximately 7.5 to 9.5 ppm) with αH and other side-chain protons. More specifically, overlay of two TOCSY spectra is shown in FIG. 1A, pink colored one is for the LaR2C peptide without RNA and the green colored one is for the La derived peptide with HCV-IRES RNA. The arrow indicates the shifting of the E177 peaks after addition of HCV-IRES RNA. All the spectra in this figure were recorded in a Bruker DRX-500 NMR spectrometer. Out of the expected 23 NH protons, 18-19 could be resolved. When sub-stoichiometric amount of RNA was added, significant shift of many protons (but not all), were observed. This suggested the existence of either an extensive peptide-RNA interface or a folding of the peptide coupled to RNA binding.

Among other shifted residues in the TOCSY spectra, one residue at 8.27 ppm showed significant shift upon complex formation (FIG. 1A; indicated by the arrow). Chemical shifts and connectivity patterns indicated that this residue is a glutamic acid (no glutamine is present in the peptide). There is only one threonine (position 5) in the peptide. Threonine α, β and methyl protons have very characteristic chemical shifts and could be easily identified. FIG. 1B shows TOCSY spectrum of the LaR2C with spin system identification of two amino acid residues, labeled with their corresponding one-letter symbols. The subscripts indicate the amino acid position in the peptide. FIG. 1C shows overlay of TOCSY (red) and NOESY (blue) spectra of the LaR2C and demonstration of TOCSY-NOESY connectivity between T178 and E177. The boxes identify the location of the NH-aH TOCSY cross peaks for the residues E177 (down field) and T178 (up field). As shown in FIGS. 1B and 1C, the position of T178. T178 was connected to the shifted glutamic acid (E177) by NOE (FIG. 1C) indicating that the glutamic acid at 8.27 ppm is E177. These results suggested that E177 is likely to be involved in recognition of HCV IRES RNA. Significant shifts were also observed corresponding to Y175 (Tyr) at the N terminus and also Y188 (Tyr) and K192-N193-E194 positions at the C terminus of the LaR2C peptide.

### Example 2

### Effect of Point Mutation within the LaR2C region of La Protein on HCV IRES Binding

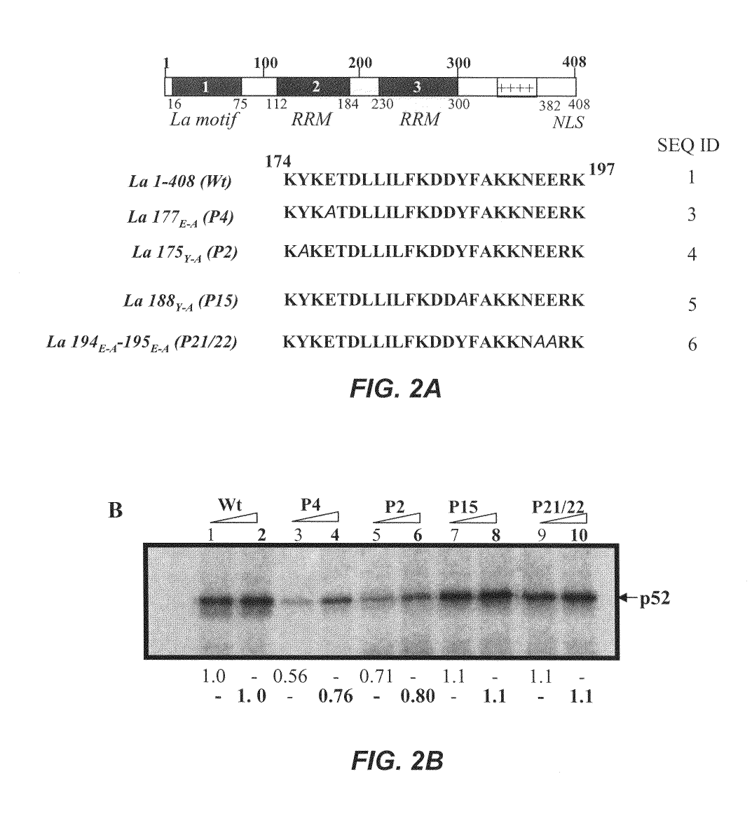

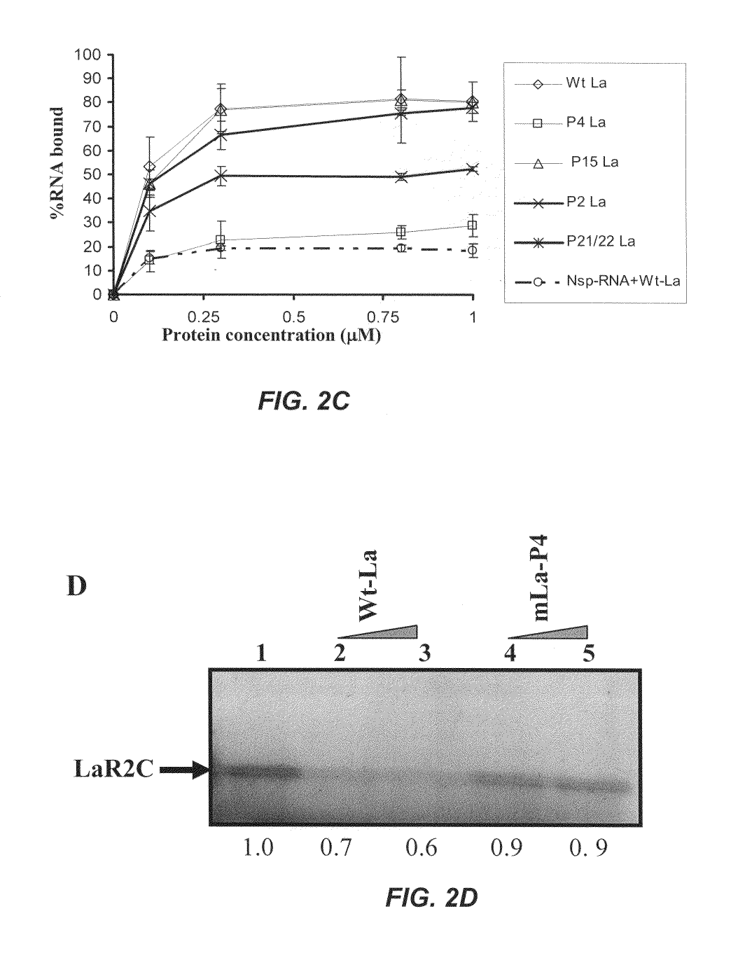

The chemical shift perturbation observed above in Example 2 may be due to direct interaction or indirect coupled folding events. In order to investigate whether the above amino acid residues of La protein were actually involved in recognition of HCV IRES RNA, corresponding point mutations in the full-length protein were generated using site-directed mutagenesis (FIG. 2A). FIG. 2A provides schematic representation of the domain organization of human La protein. The residues mutated in full-length La protein and their corresponding positions within the 24-mer LaR2C peptide (between 174-197 aa of full-length La protein) are indicated by the capital letter “P” followed by a number. For example, P4 indicates that the 4^{th} residue within the 24-mer LaR2C peptide is mutated.

The RNA binding activities of the mutant La proteins were then tested and compared with that of wildtype (Wt or wt) La protein by UV-crosslinking assay using [³²P] labeled HCV IRES RNA. More particularly, [α³²P] UTP labeled HCV IRES RNA (˜75 fmole) was UV cross-linked with increasing concentration (150, 300 ng) of either wt La protein or the mutants (as indicated on top of the lanes). The protein-nucleotide complex was resolved in SDS-10% PAGE followed by phosphor imaging analysis. The position of La protein (p52) is indicated. The band intensities corresponding to La were quantified by densitometry. The numbers below the lanes, 3, 5, 7 and 9, represent the relative intensities normalized using lane 1 (150 ng protein) as a control, whereas the numbers (in bold) below lane 4, 6, 8 and 10 represent the relative intensities using lane 2 (300 ng protein) as control. The results showed that mutations at the La175_{Y-A} and La177_{E-A} (corresponding to N-terminus P2 and P4 positions of LaR2C peptide) significantly affected the HCV IRES binding. By contrast, mutations at La188_{Y-A} and La 194_{E-A-}195_{E-A} (corresponding to C terminus, positions P15 and P21/22) did not significantly alter the RNA binding ability of La protein (FIG. 2B).

Filter binding assays were performed using [³²P] labeled HCV IRES RNA and increasing concentration of purified recombinant proteins (Wt-La or the mutants) and the results are shown in FIG. 2C. In more details, [α³²P] labeled HCV IRES RNA was bound to increasing concentrations of either wild-type La, or the mutant La proteins (as indicated). Additionally, [α³²P] labeled nonspecific RNA was also used along with the wild-type La protein. The amount of bound RNA was determined by binding to the nitrocellulose filters. The percentage of bound RNA was plotted against the protein concentration (μM). Considering the mid point of transition, mutation at the P4 residue significantly affected the RNA binding ability of La protein. Mutation at P2 residue also showed decrease in RNA binding ability, but to a lower extent when compared with the effect of the P4 mutation. Mutation at P15 or P21/22 did not alter the binding ability of La protein (FIG. 2C). As a control, a non-specific RNA probe did not show considerable binding with the wt-La protein in the same filter-binding assay.

It had previously been shown that LaR2C peptide could effectively compete against full-length La protein for binding near the iAUG within HCV IRES RNA. FIG. 2D shows the results of a competition UV-cross-linking experiment to determine the capability of various La mutants to compete against full-length La protein for RNA binding. [α³²P] UTP labeled HCV IRES RNA was pre-incubated with LaR2C peptide followed by addition of either wt-La protein (lanes 2-3) or mutant La protein (P4, lanes 4-5) in the reaction mixture for competition. The UV cross-linked complex was treated with RNase and resolved by SDS15% Tris-Tricine gel. The relative position of the band corresponding to LaR2C peptide is indicated with an arrow. The numbers below the lanes represent the relative band intensities normalized using lane 1 as a control.

As shown in FIG. 2D, full-length Wt-La protein was able to compete out binding of LaR2C with the HCV IRES RNA (FIG. 2D, lanes 2-3). By contrast, mutant P4-La protein failed to compete against the binding of LaR2C peptide effectively with the HCV IRES RNA (FIG. 2D, lanes 4-5). The result suggests that the domain of La protein encompassing the amino acid P4 (La177_{E-A}) might be involved in interacting with the HCV IRES RNA near the initiator AUG where LaR2C peptide also binds. However, binding of La protein to other sites within HCV IRES RNA might not be affected as much with the same mutation.

### Example 3

### Effect of Mutation in the LaR2C Peptide on RNA Binding and Translation

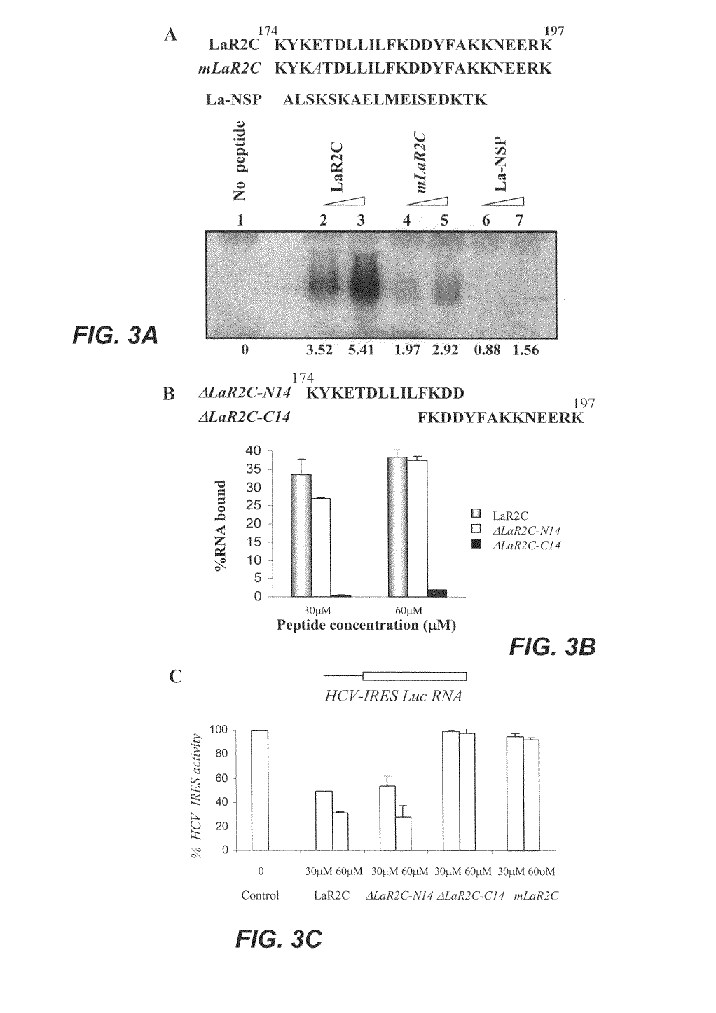

To further investigate the role of N terminal amino acids in the peptide activity, the RNA binding ability of the wild type and mutant peptide were tested by UV-cross-linking assay. The top of FIG. 3A shows schematic representation of the peptide used in the UV cross-linking analysis. The residue mutated in mutant LaR2C peptide is indicated in italics. [α³²P] UTP labeled HCV IRES RNA (˜75 fmole) was UV cross-linked with increasing concentration (30 μM, 60 μM) of LaR2C, mLaR2C and La-NSP. The peptide-nucleotide complex was resolved in 15% Tris-tricine PAGE followed by phosphor imaging analysis. The band intensities were quantified by densitometry. The numbers below the lanes represent the intensities using lane 1 (no peptide) as a control. As shown in FIG. 3A, the mutation at P4 (La177_{E-A}) greatly reduced the RNA binding ability of the mutant peptide. The non-specific peptide (Nsp) did not show any RNA binding activity (FIG. 3A).

To further confirm the RNA binding ability of the peptides, filter binding assay was performed using increasing concentration of wild-type and mutant peptide and α³²P labeled HCV IRES RNA. [³²P] labeled HCV IRES RNA was bound to increasing concentrations of either wild-type LaR2C peptide or mutant peptides as indicated in FIG. 3B. The amount of bound RNA was determined by binding to the nitrocellulose filters. The percentage of bound RNA was graphically represented against the peptide concentration (μM).

The results showed a reduced level of saturation for the mutant peptide-RNA complex compared to the wt LaR2C peptide, suggesting critical role of the P4 residue in the RNA binding activity of the LaR2C peptide (data not shown). Interestingly, deletion of N-terminal amino acids almost abrogated the RNA binding activity of the peptide (ΔLaR2C-C14), whereas retention of only 14 amino acids in the N terminus (ΔLaR2C-N14) appeared to be sufficient for significant RNA binding activity (FIG. 3B).

The effect of mutation in the LaR2C peptide was tested in an in vitro translation assays in Rabbit Reticulocyte Lysate (RRL) using uncapped monocistronic RNA containing HCV IRES upstream of Firefly luciferase gene. One microgram of uncapped HCV-IRES-Luc RNA was translated in RRL in the absence (labeled “control”) or in the presence of increasing concentration (30 and 60 μM) of either Wt LaR2C or mutant peptides (as indicated). The relative FLuc activities were represented as a percentage of the control reaction (expressed as 100%). Results showed significant decrease in HCV IRES mediated translation of HCV Luc RNA in the presence of increasing concentration (30 μM -50%, and 60 μM-70% inhibition) of Wt LaR2C peptide. By contrast, similar concentration of mutant peptide failed to inhibit the HCV IRES function significantly (FIG. 3C). Also, the C terminal truncated peptide ΔLaR2C-N14 retained the translation inhibitory activity as compared to wild-type peptide control. However, deletion of N terminal amino acids (ΔLaR2C-C14) resulted in abrogation of the translation inhibitory activity (FIG. 3C).

### Example 4

### Characterization of Conformation of the N-Terminal Seven Residue Peptide

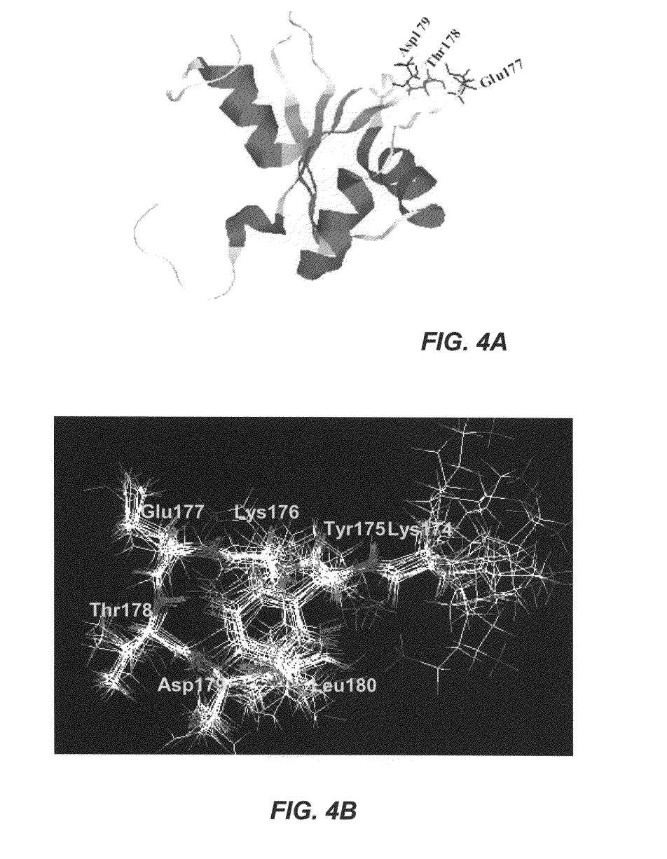

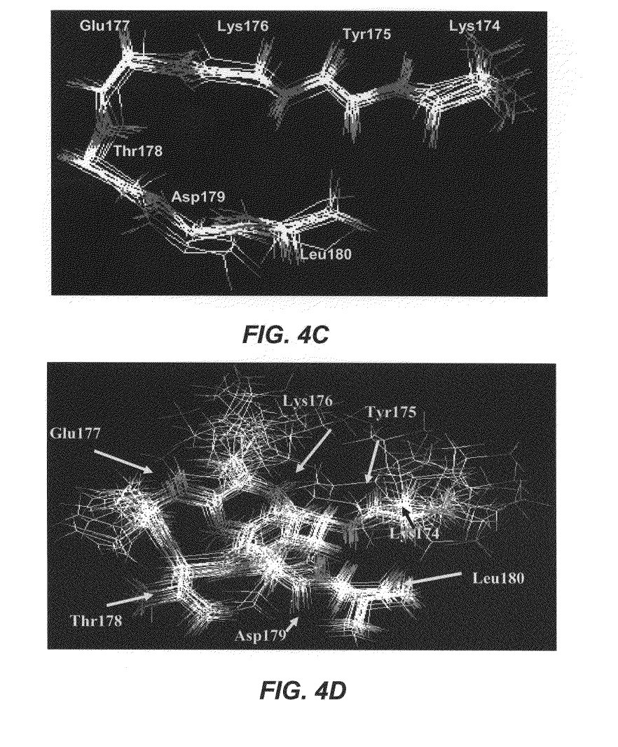

The N-terminal part of the LaR2C (174-196 aa) has been shown to constitute β4-sheet and β4′ strand of RRM (101-200). Interestingly, the residues of the peptide responsible for RNA recognition were found to map to a turn in the context of RRM (112-184) NMR structure. Based on earlier NMR structure information (PDB ID 1S79), when the LaR2C peptide was modeled, these critical N-terminal amino acids were found to be located in a similar turn that appears to be exposed for RNA binding (FIG. 4A). The helix regions are colored pink, β-sheets are colored yellow and turns and random coils are colored grey. The wt LaR2C-N7 sequence lies in the grey region (174 to 180 AA residues). The glutamic acid (177) is colored blue, threonine (178) is colored green and aspartic acid (179) is colored black.

Because the RRM (112-184) structural model suggests that the N-terminal seven residues completely cover the turn that sticks out of the globular structure of the domain (FIG. 4A), it is of interest to determine the structure and function of the 7-residue peptide.

The HCV IRES RNA bound structure of LaR2C-N7 structure was determined by NMR spectroscopy. Superimposition of best 16 structures (backbone) of 7-mer peptide (residue 174-180) under RNA bound conditions was simulated using DYANA (Version 1.5. Peter Guentert & Kurt Wuthrich, Zurich, Switzerland). The amino acid residues are represented by three letter code and numbered corresponding to their position in RRM (101-200). Under bound conditions, the peptide gave several new NOEs and change of value of J for several amide protons in addition to significant line broadening (data not shown). This indicates that conformational parameters derived from this experiment indeed reflect the bound form. The sequence KETD forms a β-turn as distance between Cα atoms of K and D is less than 7 Å (FIG. 4B). However, the Ramachandran angles do not fall within any defined turn category. The KETD sequence in the RRM (112-184) structure is also a β-turn but the conformational parameters do not fall strictly into any well-defined category (FIG. 4C). Although, the structures in two contexts are similar, there are noticeable differences in Ramachandran angles. Thus, there could be significant remodeling of the structure upon binding of La to the RNA. FIG. 4D shows 16 superimposed structures from the same region of RRM (101-200) structure for comparison. The amino acid residues are represented by three letter code and numbered from amino terminus starting from 174.

### Example 5

### The Smaller Peptide (LaR2C-N7) Inhibits HCV IRES Mediated Translation In Vitro

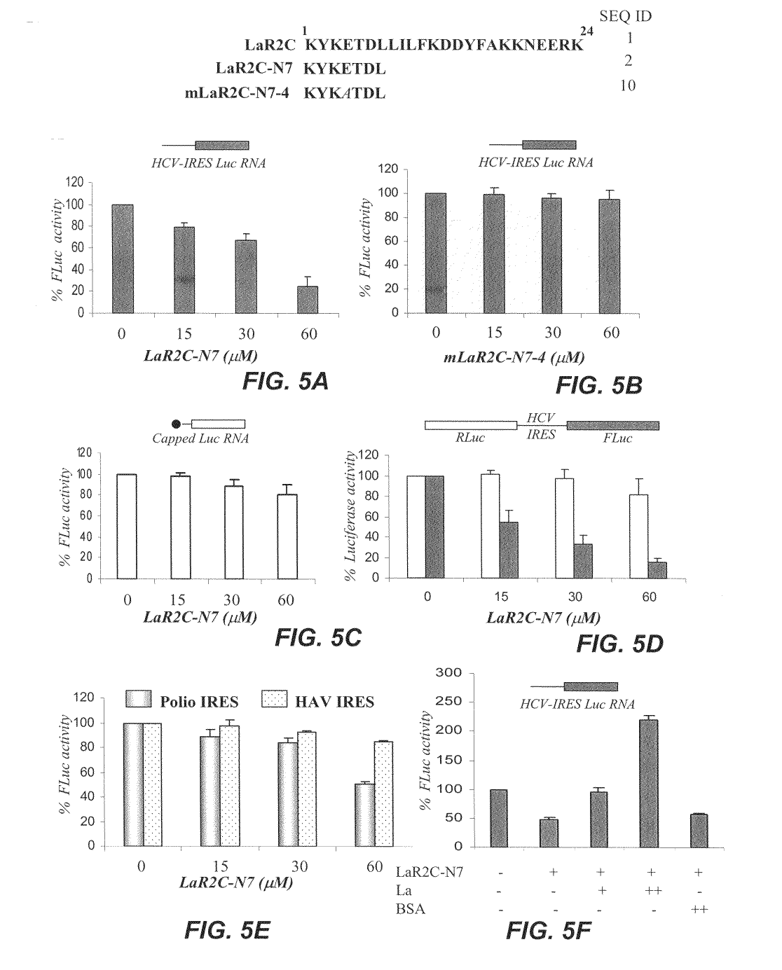

After determining the preference of beta turn conformation in the N-terminal 7-residues (“7-mer,” “N7,” or “LaR2C N7”), it was of interest to investigate whether the 7-mer peptide comprising of these amino acids would also inhibit HCV IRES mediated translation. For this purpose, monocistronic RNA containing HCV IRES upstream of reporter luciferase gene was used in the in vitro translation assays in presence or absence of increasing concentration of LaR2C-N7 peptide. The top portions of FIGS. 5A and 5B show schematic representation of the wild-type 7-mer peptide (LaR2C-N7) or the mutant 7-mer peptide along with the 24-mer wild-type LaR2C peptide. The residue mutated is indicated in italics.

The Luciferase assay was performed as follows: One microgram of uncapped HCV IRES-Luc monocistronic RNA was translated in rabbit reticulocyte lysate (RRL) in absence or presence of increasing concentration (15, 30 and 60 μM) of either the wt LaR2C-N7 or the mutant peptide mLaR2C-N7-4. Respective luciferase activities were measured and plotted against different peptide concentration. The relative FLuc activities were represented as a percentage of the control reaction (expressed as 100%). Results represent an average of three independent experiments. The smaller peptide (LaR2C-N7) showed translation inhibitory activity which is only slightly weaker than the 24-mer peptide (FIG. 5A). The IC50 for LaR2C-N7 is about 40 μM, which is slightly higher than that of LaR2C, at 30 μM (data not shown). As shown in FIG. 5B, mutation at the P4 position of the LaR2C-N7 completely abrogated its translation inhibitory activity (FIG. 5B).

The effect of LaR2C-N7 on HCV IRES mediated translation of capped RNA was investigated. One microgram of capped Luciferase RNA was translated in RRL in absence or presence of increasing concentration (15, 30, 60 μM) of LaR2C-N7 peptide and luciferase activities were plotted against different concentration of the wtLaR2C-N7 peptide. The relative FLuc activities were represented as a percentage of the control reaction (expressed as 100%). Results represent an average of three independent experiments. Interestingly, the peptide LaR2C-N7 did not show significant inhibition of capped-Luciferase RNA (representing cap-dependent translation), suggesting the specificity of the inhibition on cap-independent translation (FIG. 5C).

The effect of LaR2C-N7 on HCV IRES mediated translation in the context of HCV bicistronic RNA was also investigated. Here, one microgram of capped bicistronic RNA was translated in RRL in absence or presence of increasing concentration (15, 30, 60 μM) of LaR2C-N7 peptide and luciferase activities were plotted against different concentration of the wt LaR2C-N7 peptide. The relative RLuc and FLuc activities were represented as a percentage of the respective control reactions (expressed as 100%). Results represent an average of three independent experiments. Rluc represents cap dependent translation and Fluc represents HCV IRES mediated translation. LaR2C-N7 showed selective inhibition of HCV IRES mediated translation in the context of HCV bicistronic RNA (FIG. 5D).

The effect of LaR2C-N7 on RNA translation in the context of different viral RNA was also investigated. 1 μg of either PV-Luciferase monocistronic RNA or capped HAV-bicistronic RNA (containing Rluc-HAV-Fluc in order) translated in absence (lane 1) and presence of increasing concentrations (15, 30, 60 μM) of LaR2C-N7 peptide. PV stands for Polio virus and HAV stands for hepatitis A virus. The translation of the firefly luciferase activities were measured and plotted against the peptide concentration for the respective construct as indicated. The relative FLuc activities were represented as a percentage of the control reaction (expressed as 100%). Results represent an average of three independent experiments. The LaR2C-N7 peptide failed to inhibit IRES mediated translation of hepatitis A virus, but showed significant inhibition of Polio virus IRES function at higher concentration (FIG. 5E).

The effect of wt-La protein on the suppressive effect of LaR2C-N7 was investigated. One microgram of HCV IRES-Luc monocistronic RNA was translated in rabbit reticulocyte lysate (RRL) in absence or presence of wtLaR2C-N7 (40 μM). Increasing concentrations (25 ng, 50 ng) of purified wild-type La protein or BSA (50 ng) was added to the reactions as indicated below the lanes. Respective luciferase activities were measured and plotted in the graph. The relative FLuc activities were represented as a percentage of the control reaction (expressed as 100%). Results represent an average of three independent experiments. As shown in FIG. 5F, addition of increasing concentration purified wt-La protein showed significant rescue of the suppressive effect of LaR2C-N7 (FIG. 5F). As a control, similar concentration of BSA protein was not able to rescue the inhibition. Addition of increasing concentration of recombinant La protein (25 ng, and 50 ng) in the reaction (in absence of peptide) showed dose dependent stimulation in HCV IRES mediated translation (data not shown) as observed earlier. The result reinforces the idea that the LaR2C-N7 peptide mediated inhibition of translation may be due to competition with the endogenous La protein. Taken together, the results also suggest that the turn at the N terminus of the LaR2C peptide may be critical for its RNA binding as well as translation inhibitory activity.

### Example 6

### The Arginine-Tagged LaR2C-N7 Peptide Inhibits HCV IRES Function and Prevents Viral Replication

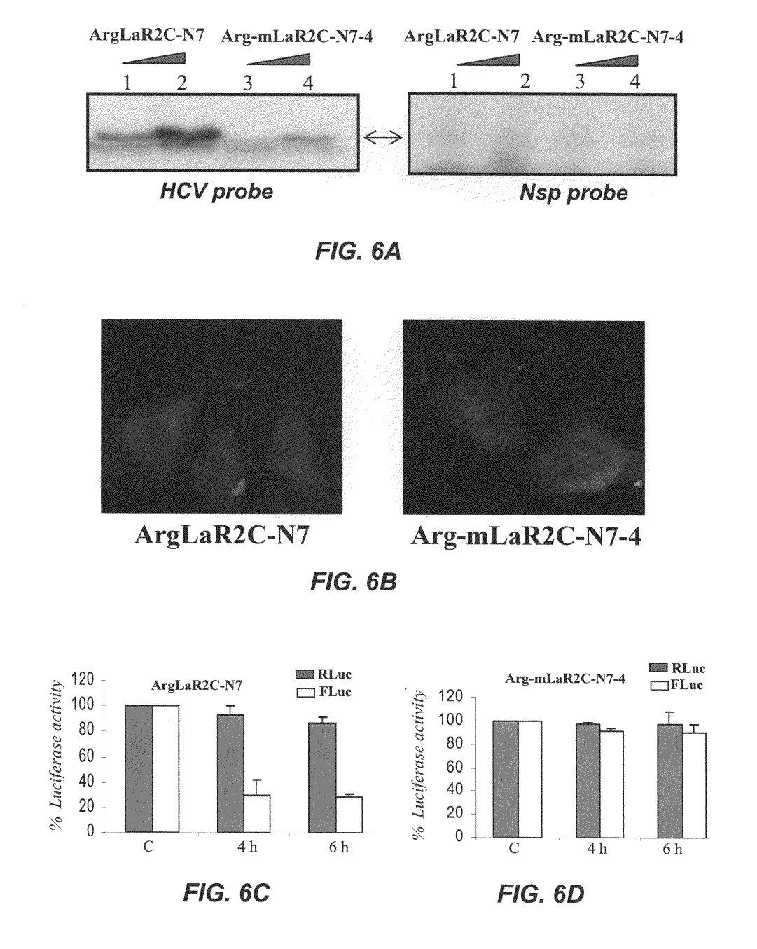

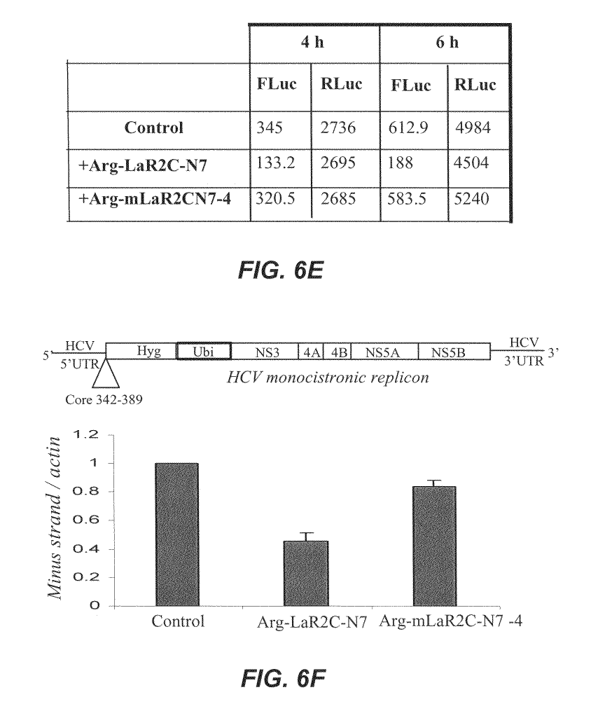

To investigate whether the LaR2C-N7 peptide would be equally effective in inhibiting HCV IRES mediated translation in Huh7 cells; we have explored delivery of the peptides inside the cells with the help of hexa-arginine fusion tag. Arginine-tagged peptide has the property to internalize into mammalian cells when supplied exogenously into the medium. The RNA binding ability of the arginine tagged LaR2C-N7 peptide was first tested by UV cross-linking experiment using [³²P] labeled HCV IRES RNA. Increasing concentrations (2 μM and 4 μM) of hexa-arginine tagged peptides, Wt Arg-LaR2C-N7 or the mutant Arg-mLaR2C-N7-4 were UV cross-linked with [α³²P] UTP labeled HCV IRES RNA or a non-specific RNA probe and analyzed in SDS-17% Tris-Tricine gel analysis followed by phosphorimaging. The arginine tagged LaR2C-N7 peptide, but not the mutant mLaR2CN7-4 (arginine-tagged) peptide, showed RNA binding activity. A non-specific RNA probe was also used as negative control in the experiment (FIG. 6A).

To investigate the internalization of the arginine-tagged peptides, fluorescein labeled hexa-arginine-tagged peptides were used. Fluorescein tagged hexa-arginine peptides (both wild-type and the mutant) were incubated with the Huh7 cells for 3 hours, extensively washed with PBS, followed by observation under a fluorescence microscope. Left panel is for Wt ArgLaR2C-N7 and right panel is mutant Arg-mLaR2C-N7-4 peptide. Both the peptides were found to be internalized inside Huh7 cells (FIG. 6B).

To investigate the effect of these peptides on HCV IRES function inside the cells, we have used these arginine-tagged peptides in Huh7 cells. Huh7 monolayer cells were first transfected with the pcDNA3-HCV bicistronic construct and incubated for 3 hours, washed and layered with medium containing 2 μM of Arg-LaR2C-N7 peptide and incubated further for either 4 hours or 6 hours. Similarly, as a negative control, another set of dishes was layered with mutant peptides (Arg-mLaR2C-N7-4). After incubation with the peptide, the cells were washed and then lysed and the luciferase activities (Fluc and Rluc) were measured. The relative luciferase activities were represented where Rluc represents cap dependent translation and Fluc represents HCV IRES mediated translation. The results showed significant decrease (inhibition up to 70%) in the HCV IRES mediated translation over the control, when cells were incubated with Arg-LaR2C-N7 peptide. However, the mutant (Arg-LaR2C-N7-4) did not show appreciable inhibitory effect (FIGS. 6C and 6D). The absolute levels of RLuc and FLuc activities of a representative experiment are presented in the table (FIG. 6E). Taken together, these results indicated that LaR2C-N7 competes with the interaction of cellular La protein to HCV IRES RNA and exert a dominant negative effect by inhibiting HCV IRES mediated translation in Huh7 cells.

To test whether this peptide would inhibit HCV replication as well, we treated Huh7 cells harboring HCV monocistronic replicon with either Wt (Arg-LaR2CN7) or mutant (Arg-mLaR2C-N7-4) peptide for 24 hours. The top portion of FIG. 6F is a schematic representation of the HCV monocistronic replicon RNA adopted from Michael et al, 2003 (Ref 26). Monolayer Huh7 cell harboring above replicon was overlaid with either Wt 7-mer (ArgLaR2C-N7) or mutant7-mer (Arg-mLaR2C-N7-4) peptide (4 μM each), added twice at 0 and 12^{th} hour. RNA was isolated at 24^{th} hour time point and subjected to cDNA synthesis. HCV negative strand was detected using real time PCR. Data were normalized with actin control and negative strand synthesis was expressed as fold change compared to control cells (in absence of peptide). The results showed almost 50% decrease in levels of HCV negative strand RNAs compared to the untreated cells when 4 μM of ArgLaR2C-N7 peptide was used (FIG. 6F). However, the mutant peptide (Arg-mLaR2C-N7-4) did not show appreciable decrease in negative strand synthesis (FIG. 6F). At lower concentration of the peptide (2 μM) the inhibition was around 30% and at higher concentration (10 μM), considerable increase in the inhibitory activity was observed (data not shown). Taken together, the results suggest that the peptide LaR2C-N7 might be effective against HCV IRES function and consequently inhibit replication of HCV RNA in Huh7 cells.

### Example 7

### Tat-N7 Fusion Protein Inhibits HCV IRES Mediated Translation and Prevents Viral Replication

This experiment was carried out to investigate whether the 7-mer peptide can inhibit the HCV IRES mediated translation in live cells when it is expressed as a fusion protein. As a first step, a polynucleotide molecule coding for the peptide LaR2C-N7 was cloned into a bacterial expression vector, pTAT, which would generate a fusion peptide containing the HIV Tat peptide and the N7. The Tat tag used here is an 11 amino acid peptide within the transduction domain of the HIV Tat protein. The presence of the Tat peptide facilitates the entry of the fusion peptide into mammalian cells when supplied exogenously into the medium. Becker-Hapak, et al., 2001.

Huh7 monolayer cells were first transfected with the pcDNA3-HCV bicistronic construct and the transfectants were selected and maintained in Neomycin containing medium. These selected cells, which constitutively express both the reporter genes, Flue and Rluc from the HCV bicistronic construct, were washed and layered with medium containing 100 nM of TAT-LaR2C-N7 fusion protein and incubated for 10 minutes. Similarly, as a negative control, another set of dishes was layered with TAT-HA protein (HA is a commonly used epitope tag). After incubation the cells were washed and incubated with fresh medium and then harvested after 6 hours. The cells were then lysed and Fluc and Rluc reporter activities were measured. The relative luciferase activities were represented as a ratio of Fluc to Rluc for normalization.

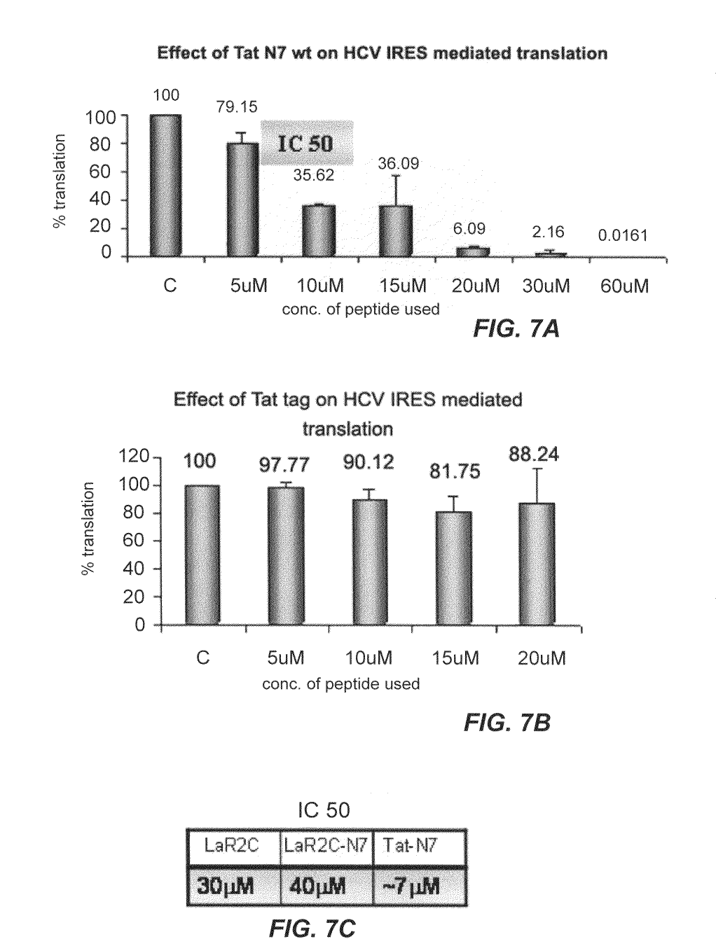

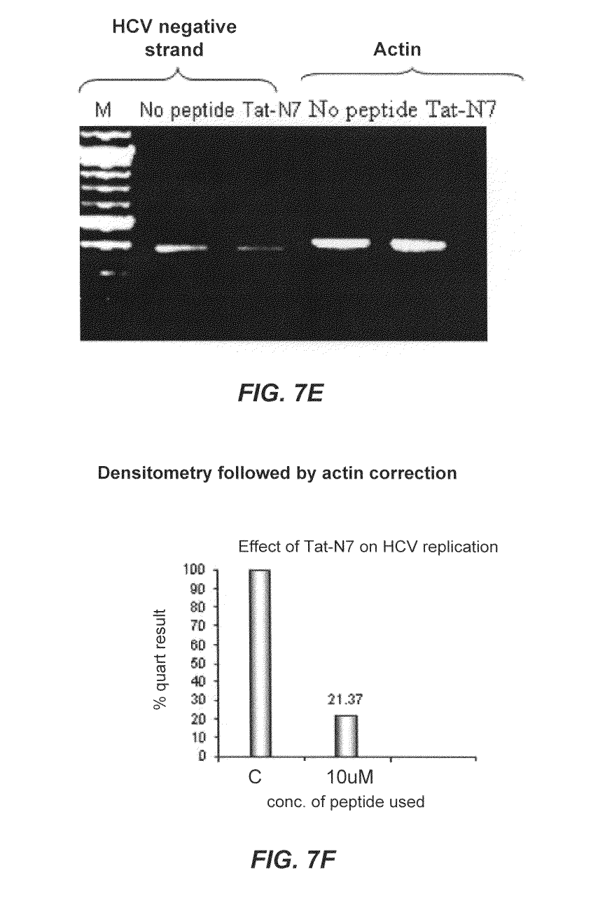

Those cells layered with TAT-LaR2C-N7 showed a decrease in the HCV IRES mediated translation over the control cells (FIG. 7A), while the TAT-HA did not have significant effect on HCV IRES mediated translation (FIG. 7B). The IC50 of TAT-LaR2C-N7 was also determined and are summarized in FIG. 7C, along with the IC50 of LaR2C and LaR2C-N7. The results indicated that, similar to the effect of LaR2C, LaR2C-N7 may also compete with the interaction of cellular La protein to HCV IRES RNA and exert a dominant negative effect by inhibiting HCV IRES mediated translation.

Because La protein has also been shown to facilitate HCV replication, it is of interest to determine if the N7-Tat peptide alters HCV replication. Ex vivo studies were performed using Huh7 cells harboring HCV replicon 2a for this study. The N7 peptide was tagged with Tat as described above.

HCV monocistronic replicon 2a construct was obtained from Dr. R Bartensclagher of Germany. FIG. 7D is a schematic representation of this vector. Huh7 cells harboring HCV replicon 2a were treated with the Tat-N7 fusion protein or actin as a negative control. The negative strand of the HCV virus was quantitated using semi-quantitative RT-PCR (FIG. 7E). As shown in FIG. 7E, the Tat-N7 fusion protein showed inhibitory effects on HCV replication, while no changes were observed for the actin control. FIG. 7F shows a densitometry quantitation of the bands shown in FIG. 7E.

### LIST OF CITED REFERENCES

The following references are incorporated by reference to the same extent as though fully replicated herein:
Ali, N., G. J. M. Pruijn, D. J. Kenan, J. D. Keene, and A. Siddiqui. 2000. Human La Antigen Is Required for the Hepatitis C Virus Internal Ribosome Entry Site-mediated Translation. J. Biol. Chem. 275: 27531-27540.
Becker-Hapak, M., S. S. McAllister, and S. F. Dowdy. 2001. TAT-Mediated protein transduction into Mammalian cells. Methods. 24: 247-56.
Choo, Q.-L., G. Kuo, A. J. Weiner, L. R. Overby, D. W. Bradley, and M. Houghton 1989. Isolation of a cDNA clone derived from a blood-borne non-A, non-B viral hepatitis genome. Science. 244: 359-362.
Costa-Mattioli, M., Y. Svitkin, and N. Sonenberg. 2004. La autoantigen is necessary for optimal function of the poliovirus and hepatitis C virus internal ribosome entry site in vivo and in vitro. Mol. Cell. Biol. 24: 6861-6870.
Das, S., M. Ott, A. Yamane, W. Tsai, M. Gromier, F. Lahser, S. Gupta, and A. Dasgupta. 1998. A Small Yeast RNA Blocks Hepatitis C Virus Internal Ribosome Entry Site (HCV IRES)-Mediated Translation and Inhibits Replication of a Chimeric Poliovirus under Translational Control of the HCV IRES Element. J. Virol. 72: 5638-5647.
Houghton, M., Weiner, A., Han, J., Kuo, G., and Choo, Q.-L. (1991) Hepatology 14, 381-388.
Izumi, R. E., S. Das, B. Barat, S. Raychaudhuri, and A. Dasgupta. 2004. A peptide from autoantigen La blocks poliovirus and hepatitis C virus cap-independent translation and reveals a single tyrosine critical for La RNA binding and translation stimulation. J. Virol. 78: 3763-3776.
Kato, N., Hijikata, M., Ootsuyama, Y., Nakagawa, M., Ohkoshi, S., Sugimura, T., and Shimotohno, K. (1990) Proc. Natl. Acad. Sci. U.S.A. 87, 9524-9528.
Lee C C, Chiang B L., RNA interference: new therapeutics in allergic diseases. Curr Gene Ther. (2008)8(4):236-46.
Nazari R, and Joshi S., CCR5 as Target for HIV-1 Gene Therapy. Curr Gene Ther. (2008) 8(4):264-72.
Pudi, R., S. Abhiman, N. Srinivasan, and S. Das. 2003. Hepatitis C Virus Internal Ribosome Entry Site-mediated Translation Is Stimulated by Specific Interaction of Independent Regions of Human La Autoantigen. J. Biol. Chem. 278:12231-12240.
Pudi, R., P. Srinivasan, and S. Das. 2004. La Protein Binding at the GCAC Site Near the Initiator AUG Facilitates the Ribosomal Assembly on the Hepatitis C Virus RNA to Influence Internal Ribosome Entry Site-mediated Translation. J. Biol. Chem. 279: 29879-29888.
Spahn, C. M. T, Kieft, J. S., Grassucci, R. A., Penczek, P. A., Zhou, K., Doudna, J. A and Frank, J. (2001) Hepatitis C virus IRES RNA-induced change in the conformation of the 40S ribosomal subunit. Science, 291, 1959-1962.
Tsukiyama-Kohara, Z., N. Iizuka, M. Kohara, and A. Nomoto. 1992. Internal ribosome entry site within hepatitis C virus RNA. J. Virol. 66: 1476-1483.
Otto G. A., and J. D. Puglisi. 2004. The pathway of HCV IRES-mediated translation initiation. Cell. 119(3): 369-80.

1. A composition comprising an HCV inhibiting agent, wherein said HCV inhibiting agent is capable of competing against the La protein for binding to HCV IRES RNA and thereby inhibiting the translation of HCV RNA mediated by the La protein, and said HCV inhibiting agent comprises a peptide having less than 24 amino acids.

2. The composition of claim 1, wherein the HCV inhibiting agent comprises a peptide having a sequence at least 70% identical to the sequence of SEQ ID NO. 2.

3. The composition of claim 1, wherein said peptide is part of a fusion protein.

4. The composition of claim 1, wherein the HCV inhibiting agent comprises a peptide the peptide has a sequence identical to SEQ ID NO. 2.

5. The composition of claim 1, further comprising at least one ingredient selected from the group consisting of a diluent, an adjuvant, and a carrier.

6. A composition comprising an HCV inhibiting agent, wherein said HCV inhibiting agent is capable of competing against the La protein for binding to HCV IRES RNA and thereby inhibiting the translation of HCV RNA mediated by the La protein, and said HCV inhibiting agent comprises a peptide having less than 24 amino acids and having a sequence at least 70% identical to the sequence of SEQ ID NO. 2.

7. A polynucleotide comprising the nucleic acid sequence encoding a peptide having the sequence of SEQ ID NO. 2.

8. An expression vector comprising the polynucleotide of claim 7.

9. The expression vector of claim 8, further comprising a promoter operably linked to the coding sequence of said peptide.

10. An antiviral agent comprising a molecule, said molecule having a structure similar to the turn structure exhibited in solution by a peptide having the sequence of SEQ ID NO. 2, wherein said agent is capable of effectively blocking the ribosome assembly on the HCV RNA and said molecule is selected from the group consisting of a peptide, a polynucleotide, and an organic molecule.

11. The antiviral agent of claim 10, wherein the molecule is a peptide.

12. The antiviral agent of claim 10, wherein the molecule is an organic molecule.

13. A composition comprising an HCV inhibiting agent, said HCV inhibiting agent being capable of competing against the La protein for binding to HCV IRES RNA and thereby inhibiting the translation of HCV RNA mediated by the La protein, wherein the IC50 for said inhibition is not more than 100 μM.

14. The composition of claim 13, wherein the IC50 is in the range of from about 10 μM to 50 μM.

15. A method for inhibiting or preventing viral infection in a host, comprising the steps of:
(a) administering to said host a composition comprising an HCV inhibiting agent,
(b) allowing said HCV inhibiting agent to compete against La protein endogenous to the host for binding to HCV IRES RNA, and
(c) inhibiting the translation of HCV RNA mediated by the La protein,
wherein the HCV inhibiting agent is selected from the group consisting of a peptide having less than 24 amino acids and having a sequence at least 70% identical to the sequence of SEQ ID NO. 2, a polynucleotide encoding a peptide having less than 24 amino acids and having a sequence at least 70% identical to the sequence of SEQ ID NO. 2, and an organic molecule having a structure similar to the turn structure exhibited in solution by a peptide having the sequence of SEQ ID NO. 2.

16. The method of claim 15 wherein the viral infection is hepatitis C viral infection.

17. The method of claim 15 wherein the HCV inhibiting agent is a peptide.

18. The method of claim 17 wherein the peptide has a sequence identical to SEQ ID NO. 2.

19. The method of claim 15 wherein the HCV inhibiting agent is a polynucleotide, wherein said polynucleotide is expressed as a peptide in the host, said peptide being capable of competing against the La protein endogenous to the host for binding to HCV IRES RNA, thereby inhibiting the translation of HCV RNA in said host.

20. The method of claim 15 wherein the HCV inhibiting agent further comprises at least one ingredient selected from the group consisting of a diluent, an adjuvant, and a carrier.


SEQUENCE LISTING (2 sequences)

SEQ ID NO: 1 - protein, 24 aa, Homo sapiens
KYKETDLLILFKDDYFAKKNEERK

SEQ ID NO: 2 - protein, 7 aa, Homo sapiens
KYKETDL